(19) United States
(12) Patent Application Publication
(10) Pub. No.: US 2006/0019282 A1
(43) Pub. Date: Jan. 26, 2006

(54) Method and unit for detecting an interaction such as hybridization, bioassay plate provided with a number of such detecting units, system for detecting an interaction such as hybridization, and reagent kit

(76) Inventor: Makiko Negishi, Tokyo (JP)

Correspondence Address: William S. Frommer, Esq.;FROMMER LAWRENCE & HAUG LLP, 745 Fifth Avenue, New York, NY, 10151, US

(21) Appl. No.: 11/167,738

(22) Filed: Jun. 27, 2005

(30) Foreign Application Priority Data
Jun. 29, 2004 (JP) 2004-191047

(51) Int. Cl. C12Q 1/68

(52) U.S. Cl. 435/6

(57) ABSTRACT

Disclosed is a method for the detection of an interaction between substances. According to the method, a salt-containing HEPES buffer is allowed to exist in a reaction region that provides a place of interaction for the interaction. The concentration of the salt in the salt-containing HEPES buffer may be adjusted such that one or more substances each having a negative charge, such as a probe nucleic acid, target nucleic acid and/or intercalator, can be prevented from undergoing non-specific adsorption on a positively-charged surface of a solid phase. Also disclosed are an interaction detecting unit including the reaction region and a medium existing in the reaction region and including the salt-containing HEPES buffer; a bioassay plate including a DNA chip provided with a number of such interaction detecting units; a system for detecting an interaction such as hybridization; and a reagent kit including the salt-containing HEPES buffer.

### BACKGROUND OF THE INVENTION

This invention relates to an interaction detecting technique making use of a salt-containing HEPES buffer. More specifically, the present invention is concerned with a method and unit for detecting an interaction such as hybridization, a bioassay plate provided with a number of such detecting units, a system for detecting an interaction such as hybridization, and a reagent kit, all of which make use of a salt-containing HEPES buffer.

Nowadays, bioassay plates such as DNA chips (or DNA microarrays) are used in mutation analyses of genes, SNPs (single-base polymorphisms) analyses, gene expression frequency analyses, and the like, and have begun to find utility in a wide variety of fields such as drug developments, clinical diagnoses, pharmacogenomics and forensic medicine.

The term “bioassay plate” as used herein means a glass, silicon or like substrate with a wide variety of numerous detecting or probe substances (hereinafter referred to as probe substances) integrated and immobilized at a high density thereon. With the wide variety of numerous probe substances immobilized on the substrate, a comprehensive analysis can be performed on one or more target substances that interact with the corresponding ones of the probe substances.

As an illustrative method for the immobilization of probe substances on a substrate, an avidin-coated, solid-phase surface can be arranged in each reaction region on the substrate. By inducing the formation of an avidin-biotin linkage between the avidin layer arranged in the reaction region and its corresponding biotin-modified probe substance, the probe substance can be efficiently immobilized on the substrate.

In the case of a DNA chip or the like, an intercalator may be used for the detection of hybridization (an interaction between a probe nucleic acid and a target nucleic acid). This intercalator can insert and bind itself to a double-stranded nucleic acid or the like and therefore, can be used for the detection of hybridization.

Japanese Patent Laid-open No. 2003-84002 discloses a method for reducing a background noise by using a quencher upon detection of fluorescence on a DNA chip. Japanese Patent Laid-open No. 2004-24035, 2002-181816, Hei 11-164700, on the other hand, each discloses a method for detecting a nucleic acid by using an intercalator.

### SUMMARY OF THE INVENTION

In the past detection of an interaction by the use of a bioassay plate involves a problem in that substances having a negative charge, such as nucleic acids, nonspecifically adsorb to a positively-charged surface of a solid phase in each reaction region. Some intercalators may also nonspecifically adsorb to the positively-charged surface of the solid phase in each reaction region. There is an outstanding need for the reduction of nonspecific adsorption, because the nonspecific adsorption of such a nucleic acid or intercalator results in a background noise upon detection of hybridization or the like.

Further, the binding characteristics of an intercalator can be hardly retained for a long time. It has, therefore, been necessary to detect hybridization at an early stage after adding the intercalator to a reaction region.

In addition, an intercalator also binds to some molecules of a single-stranded nucleic acid. It has, therefore, been necessary to conduct a washing step before the detection of hybridization so that the unhybridized, single-stranded nucleic acid molecules can be removed.

Therefore, it is desirable to implemant the prevention of nonspecific adsorption of a nucleic acid and/or an intercalator to a surface of a solid phase in a reaction region, the long-hour retention of the binding characteristics of an intercalator for use in the detection of hybridization, and the simplification of a washing step required upon detection of hybridization.

The present invention, therefore, provides (1) an interaction detecting method, (2) a hybridization detecting method, (3) an interaction detecting unit, (4) a bioassay plate, (5) an interaction detecting system, (6) a hybridization detecting unit, (7) a DNA chip, (8) a hybridization detecting system, and (9) a reagent kit. They will hereinafter be described in order.

(1) Interaction Detecting Method

The interaction detection method according to the present invention includes allowing a HEPES buffer, which contains a salt, to exist in a reaction region that provides a place of interaction for an interaction between substances.

To immobilize a probe substance in a reaction region on a substrate via an avidin-biotin linkage, for example, a positively-charged solid-phase surface (avidin layer) is arranged in the reaction region. When the positively-charged solid-phase surface is arranged in the reaction region, substances, an intercalator and the like, each of which has a negative charge, may nonspecifically adsorb. The present invention prevents the nonspecific adsorption of substances, each of which has a negative charge, to a positively-charged solid-phase surface by adjusting the concentration of the salt in the salt-containing HEPES buffer.

The preventive effect on the nonspecific adsorption of substances, each of which has a negative charge, by the adjustment of the concentration of the salt in the salt-containing HEPES buffer is presumably attributed to ionic shielding or concentration of counter ions. Described specifically, the preventive effect is considered to be brought about in the following mode of action mechanism.

The adjustment of the concentration of the salt in the salt-containing HEPES buffer results in excessive existence of cations and anions in the HEPES buffer. The excessively-existing cations loosely surround the substances, each of which has the negative charge, so that the substances are shielded from the positively-charged solid-phase surface. On the other hand, the anions and the positively-charged solid-phase surface attract each other, so that the anions loosely cover the solid-phase surface to also shield the solid-phase surface from the substances each of which has the negative charge. Accordingly, the substances each of which has the negative charge are considered to be prevented from nonspecifically adsorbing to the positively-charged solid-phase surface owing to the shielding of the solid-phase surface and the substances from each other by the adjustment of the concentration of the salt in the salt-containing HEPES buffer.

It is to be noted that no particular limitation is imposed on the substances each of which has the negative charge insofar as they nonspecifically adsorb to the positively-charged solid-phase surface. For example, probe nucleic acids (nucleic acids useful as probe substances), target nucleic acids (nucleic acids as target substances) and the like are included in substances each of which has a negative charge.

(2) Hybridization Detecting Method

The hybridization detecting method according to the present invention performs the detection of hybridization between a probe nucleic acid and a target nucleic acid by using an intercalator, and is devised to allow a salt-containing HEPES buffer to exist in a reaction region that can provide a place of hybridization.

The use of the salt-containing HEPES buffer makes it possible to prevent the nonspecific adsorption of the intercalator to the positively-charged solid-phase surface.

In addition, the use of the salt-containing HEPES buffer in the detection of hybridization by employing the intercalator can maintain the structural stability of the intercalator used for the detection of a double-stranded nucleic acid (including the hybridized one) and therefore, can inhibit changes in the binding characteristics of the intercalator to the double-stranded nucleic acid. Further, the use of the salt-containing HEPES buffer can increase the quantity of fluorescence from the intercalator, and can also maintain the quantity of fluorescence from the intercalator for a long time without a reduction.

The use of the salt-containing HEPES buffer can make greater the quantity of fluorescence from the intercalator upon its binding or the like to a double-stranded nucleic acid in comparison with the quantity of fluorescence from the intercalator upon its binding or the like to a target nucleic acid in the form of a single strand. Even when a high-temperature condition is applied, the fluorescence ratio of the quantity of fluorescence from the intercalator upon its binding or the like to the double-stranded nucleic acid to the quantity of fluorescence from the intercalator upon its binding or the like to the target nucleic acid in the form of the single strand can still be maintained at a large value.

The use of the salt-containing HEPES buffer can maintain the quantity of fluorescence from the intercalator at a high level even when the temperature is raised inside the reaction region or the pH of the salt-containing HEPES buffer varies as a result of a change in the temperature inside the reaction region.

The use of the salt-containing HEPES buffer can also reduce a change in the pH inside the reaction region, which takes place as a result of a temperature change. Described specifically, even when a condition of a large temperature change range is applied, the pH change inside the reaction region can be controlled within a range in which the binding characteristics, fluorescence quantity and the like of the intercalator can be maintained without substantial changes.

Based on the above-described actions and effects of the salt-containing HEPES buffer, the present invention provides a method for detecting hybridization, which includes a procedure of controlling the temperature of a reaction region, in which a salt-containing HEPES buffer and an intercalator are allowed to exist, at a temperature suited for the hybridization. Control of the temperature inside a reaction region, for example, at a temperature suited for hybridization upon detection of the hybridization can facilitate the promotion of the hybridization and the shortening of a time required for the detection of the hybridization.

The present invention also provides a method for detecting hybridization, wherein a salt-containing HEPES buffer has an acidic pH. According to this method, the binding characteristics, fluorescence quantity and the like of an intercalator can be retained to permit the detection of hybridization even when the pH of the buffer in the reaction region falls within an acidic range lower than 7.0, for example, as a result of a temperature adjustment upon detection of the hybridization.

The present invention also provides a method for detecting hybridization, wherein an intercalator is added into a reaction region, for example, by dropping, injection or the like at one of the following stages (1) to (3):

(1) a pre-stage at which a target nucleic acid is added to a salt-containing HEPES buffer in the reaction region,

(2) a stage at which a probe nucleic acid is added to the salt-containing HEPES buffer in the reaction region, and

(3) a stage at which the hybridization proceeds.

It is to be noted that the stage (1) means a stage preceding the addition of the target nucleic acid to the HEPES buffer in the reaction region and including, for example, a stage at which the HEPES buffer is added to the reaction region, for example, by dropping, injection or the like. It is also to be noted that the stage (3) means a stage up to the completion of the hybridization and includes, for example, a stage at which the hybridization has begun to proceed by the addition of a target nucleic acid, a stage at which conditions of a predetermined time and a predetermined temperature are applied to promote the progress of the hybridization, and a stage up to the completion of the hybridization subsequent to the addition of the above-described temperature conditions or the like.

As mentioned above, the use of a salt-containing HEPES buffer makes it possible to retain the binding characteristics of an intercalator for a long time. It is, therefore, still possible to detect hybridization even when a predetermined time has elapsed with the intercalator kept placed within the reaction region. Even when the temperature or the pH of the HEPES buffer changes, the binding characteristics of the intercalator can be retained. It is hence possible, for example, to apply a condition of raising the temperature of the HEPES buffer to around Tm or to a temperature suited for hybridization or annealing, as needed, with the intercalator kept added within the reaction region. It is, therefore, possible to achieve an improvement in the detection accuracy and also to make the procedure or steps optimal and efficient.

The use of the salt-containing HEPES buffer can also make greater the quantity of fluorescence from the intercalator upon its binding or the like with a double-stranded nucleic acid in comparison with the quantity of fluorescence from the intercalator upon its binding to a target nucleic acid in the form of a single strand. The double-stranded nucleic acid (hybridization) can, therefore, be detected even when some molecules of the single-stranded nucleic acid still remains unhybridized in the reaction region. It is, accordingly, possible to omit or simplify the step of washing off the unhybridized, single-stranded nucleic acid molecules.

Further, the intercalator may be added to the reaction region concurrently with the HEPES buffer or the target nucleic acid at the stage that the HEPES buffer is added to the reaction region by dropping, injection or the like or at the stage that the target nucleic acid is added to the HEPES buffer in the reaction region. This concurrent addition of the intercalator can omit or simplify the procedure of separately adding the intercalator.

The addition of the intercalator to the reaction region at one of the above-described stages has another advantage in that the time required for the detection can be shortened, because the intercalation and the hybridization can be conducted together as a single step.

(3) Interaction Detecting Unit, (4) Bioassay Plate, and (5) Interaction Detecting System

The present invention also provides an interaction detecting unit including a reaction region for providing a place of interaction for an interaction between substances, and a medium existing in the reaction region and including a salt-containing HEPES buffer; a bioassay plate including a number of such interaction detecting units; and an interaction detecting system including temperature control means for controlling a temperature in the reaction region of the interaction detecting unit, and means for detecting the interaction. In these unit, plate and system, the above-mentioned mode of action mechanism, actions and effects of the salt-containing HEPES buffer can also be used effectively. No particular limitation is imposed on the interaction detecting means.

(6) Hybridization Detecting Unit, (7) DNA Chip, and (8) Hybridization Detecting System

The present invention also provides a hybridization detecting unit including a reaction region for providing a place of hybridization for hybridization between a probe nucleic acid and a target nucleic acid, and a medium existing in the reaction region and including a salt-containing HEPES buffer and an intercalator; a DNA chip including a number of such hybridization detecting units; and a hybridization detecting system including temperature control means for controlling a temperature in the reaction region of the hybridization detecting unit, and fluorescence detecting means for having the intercalator fluorescently excited to emit fluorescence and detecting an intensity of the fluorescence. In these unit, chip and system, the above-mentioned mode of action mechanism, actions and effects of the salt-containing HEPES buffer can also be used effectively.

(9) Reagent Kit

The present invention also provides a reagent kit including at least one of a salt-containing HEPES buffer, which has been prepared for a detection of an interaction between substances, and a composition useful for the preparation of the salt-containing HEPES buffer. When the interaction is hybridization, this reagent kit may further include, for example, an intercalator for use in the detection of the hybridization. The combination of such reagents into a kit can obviate the procedure which would otherwise be required for the preparation of the reagents, thereby making it possible to simplify the procedure of the entire assay.

It is to be noted that the individual technical terms as used herein can be defined as will be described hereinafter.

The term “HEPES” means 2-[4-(2-hydroxyethyl)-1-piperazinyl]ethanesulfonic acid, which is one of Good buffer substances or reagents.

The term “nucleic acid” means a polymer (nucleotide chain) of the phosphate ester of a nucleoside composed of purine or a pyrimidine residual group and a saccharide coupled together via a glycosidic linkage, and embraces therein a wide variety of nucleotide chains such as oligonucleotides including probe DNAs, polynuleotides, DNAs (whole lengths and their fragments) formed of purine nucleotides and pyrimidine nucleotides polymerized with each other, and cDNAs, RNAs and PNAs (polyamide nucleotide derivatives) obtained by reverse transcription.

The term “probe substance” means a substance immobilized or liberated beforehand in a reaction region and adapted to function as a probe for detecting a substance which specifically interacts with the substance. The term “target substance” means a substance which specifically interacts with the probe substance.

The term “probe nucleic acid” indicates a case in which the probe substance is a nucleic acid, while the term “target nucleic acid” indicates a case in which the target substance is a nucleic acid. In these cases, the term “interaction” means hybridization.

The term “reaction region” means a region or space that can provide a place of reaction for hybridization. Illustrative is a place of reaction in the form of a well in which a liquid phase, gel or the like can be stored.

The term “hybridization” means a complementary chain (double strand) forming reaction, which like the linkage between a probe nucleic acid and a target nucleic acid, takes place between nucleic acids equipped with complementary base sequence structures.

The term “solid-phase surface” means a surface of a solid material such as a substrate or beads, which is a critical surface relative to a region or space which can provide a place of reaction for hybridization or another interaction.

The term “intercalator” is a substance which can insert and bind itself to a double-stranded nucleic acid or the like, or a composition containing the substance. When an intercalator is fluorescence-labeled or is a fluorescent substance itself, the existence of a double-stranded nucleic acid (hybridization) can be confirmed from fluorescence. Illustrative intercalators capable of detecting double-stranded nucleic acids from fluorescence include POPO-1, TOTO-1, SYBRGreen and PicoGreen.

The present invention can prevent the nonspecific adsorption of a nucleic acid, an intercalator and/or the like to the surface of a solid phase in a reaction region. The present invention can also maintain for a long time the binding characteristics of an intercalator for use in the detection of hybridization.

The methods according to the present invention can retain the binding characteristics of an intercalator for a long time, and further, can improve the accuracy of a detection of hybridization. The methods according to the present invention are, therefore, useful in the field of bioassay plates such as DNA chips. The methods according to the present invention are also believed to have high general utility and high utility value in that they can simplify the washing step upon detection of hybridization. The methods according to the present invention have a still further advantage in that the background noise can be reduced upon detection or the like of hybridization.

The interaction detecting units according to the present invention are useful in a bioassay plate such as a DNA chip. The bioassay plate, such as the DNA chip, and interaction detecting system according to the present invention are useful for the detection of an interaction such as hybridization. In addition, the combination of reagents, which are used in the above-described methods, into a kit can simplify the preparation procedure of the reagents. The present invention is, therefore, believed to be useful in industry.

### BRIEF DESCRIPTION OF THE DRAWINGS

FIG. 1 is a cross-sectional view of a suitable embodiment of an interaction detecting unit, which can implement the present invention;

FIGS. 2A through 2C are schematic cross-sectional views of the interaction detecting unit of FIG. 1, and illustrate an embodiment of an interaction detecting method (including a hybridization detecting method) according to the present invention at different stages;

FIG. 3 is a perspective view of an embodiment of a bioassay plate according to the present invention;

FIG. 4 is a block diagram of an embodiment of an interaction detecting system according to the present invention; and

FIG. 5 is a graphic representation of the pHs of buffers as a function of their temperatures.

### DETAILED DESCRIPTION OF THE PREFERRED EMBODIMENTS

With reference to the accompanying drawings, certain preferred embodiments of the present invention will be described hereinafter.

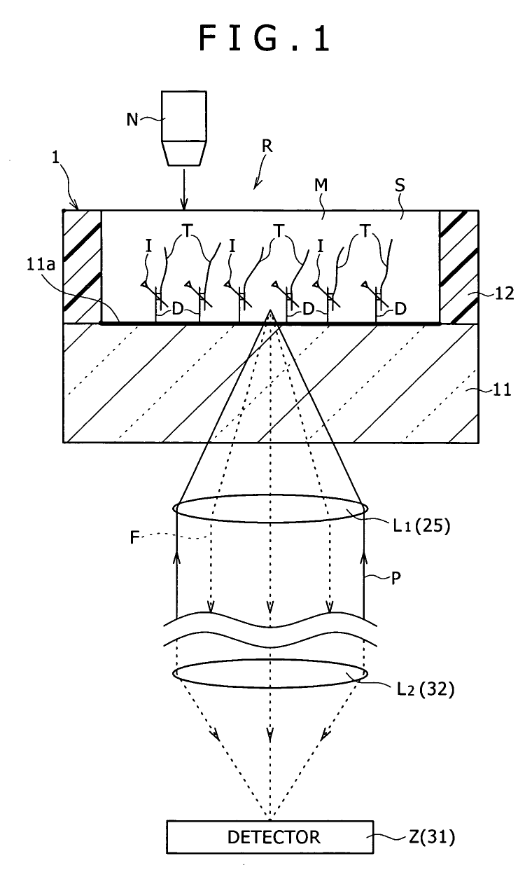

FIG. 1 is a cross-sectional view schematically illustrating one of interaction detecting units arranged on a substrate. Numeral 1 shown in FIG. 1 designates a typical example of the embodiment of the interaction detecting unit according to the present invention. Use of this interaction detecting unit 1 makes it possible to practice the interaction detecting method according to the present invention, which can be the hybridization detecting method according to the present invention.

The interaction detecting unit 1 is provided with a substrate layer 11 and a reaction-region forming layer 12 forming a reaction region R that presents a well-like configuration. Into the reaction region R, a salt-containing HEPES buffer S, a target nucleic acid T, an intercalator I and the like can be dropped or injected through nozzles N such as inkjet printing nozzles. It is to be noted that by way of example, FIG. 1 shows a state in which molecules of a probe nucleic acid D are immobilized at one ends thereof in the reaction region R and molecules of the intercalator I are inserted in and bound to sites of base pairs in molecules of a double-stranded nucleic acid formed by hybridization.

The substrate layer 11 is formed, for example, with quartz glass, silicon, or a synthetic resin such as a polycarbonate or polystyrene. The substrate layer 11 may preferably be made of a material which permits transmission of excitation light P of a predetermined wavelength (for example, fluorescent excitation light). When hybridization is fluorescently detected by using an intercalator, for example, the formation of the substrate layer 11 with a material which permits transmission of the excitation light P makes it possible to detect the hybridization in the reaction region R by irradiating the excitation light P from a lower side of the interaction detecting unit 1.

The reaction-region forming layer 12 can be formed, for example, with a photosensitive polyimide resin. By subjecting this photosensitive polyimide resin to surface treatment while making use of a photoresist, a number of fine reaction regions R can be formed.

The reaction region R allows a medium M to exist therein, specifically can store or hold it. No particular limitations are imposed on its configuration and size, but its length, width and depth may each range from several micrometers to several hundred micrometers. These dimensions can be determined based on the spot diameter of the excitation light P and the possible minimum drop volume of the medium M such as a sample solution (a solution with a probe nucleic acid contained therein, a solution with a target nucleic acid contained therein).

The medium M contains the salt-containing HEPES buffer S. Owing to the inclusion of the salt-containing HEPES buffer S, a probe substance D, the target substance T and the intercalator I having the negative charge, all of which had been dropped or injected into the medium M, can be effectively prevented from nonspecifically adsorbing to the positively-charged solid-phase surface. No particular limitation is imposed on the salt in the salt-containing HEPES buffer S, although NaCl, MgCl₂ or the like is suited. The salt-containing HEPES buffer S can preferably have such a salt concentration as giving an ionic strength sufficient to prevent nonspecific adsorption of a substance, which has a negative charge, to a positively-charged solid-phase surface, and more specifically, can have such a salt concentration as giving an ionic strength equivalent to at least 20 mM of MgCl₂.

Even when a condition of a wide range of temperature changes is applied upon detection of hybridization by using the intercalator I that inserts and binds itself to the base pair of a complementary chain, pH changes in the reaction region R can be controlled within such a range that the binding characteristics, fluorescence quantity and the like of the intercalator can be maintained without substantial changes. It is, therefore, possible to surely control the interior of the reaction region R at a temperature suited for hybridization.

Even when the pH of the buffer in the reaction region drops to an acidic range below 7.0 as a result of a temperature control upon detection of hybridization, the binding characteristics and fluorescence quantity of the intercalator I can be retained without substantial changes. The salt-containing HEPES buffer S in the medium M may, therefore, have an acidic pH.

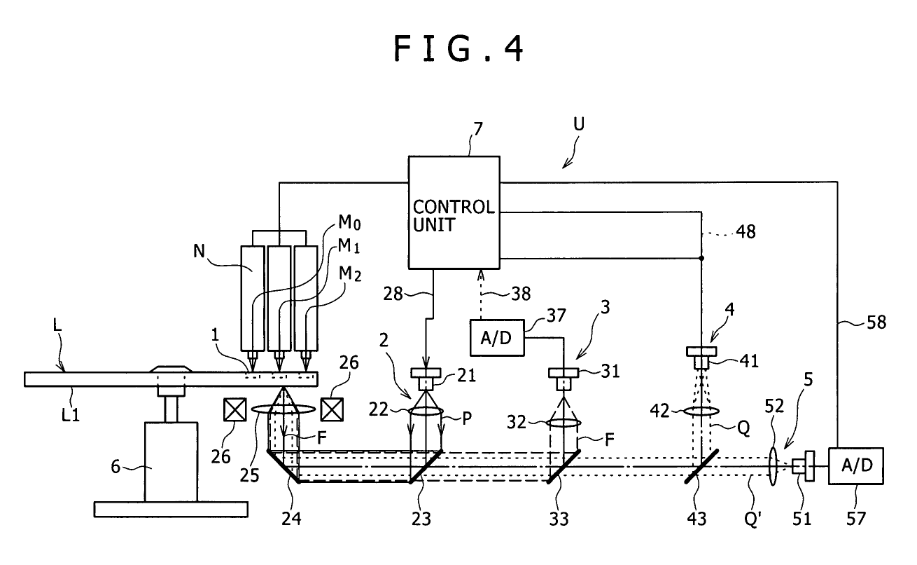

In FIG. 1, sign P indicates excitation light, while sign F designates fluorescence emitted as a result of excitation by the excitation light P. The fluorescence F, which has been emitted as a result of excitation by the excitation light P entered the reaction region R from the side of a back surface of the light-transmitting substrate layer 11, travels through a lens L₁ and a condenser lens L₂. The lens L₁ converts the fluorescence F into parallel rays and is equivalent to numeral 25 in FIG. 4, while the condenser lens L₂ condenses the fluorescence F and is equivalent to numeral 32 in FIG. 4. The intensity of the fluorescence F can be detected by a detector Z which is equivalent to numeral 31 in FIG. 4.

It is to be noted that FIG. 1 illustrates as a representative example the case in which the interaction is hybridization and the intercalator I is used as means for detecting the hybridization. It is also to be noted that the present invention shall not be limited narrowly to this example.

In FIG. 1, the substrate layer 11 is provided with a solid-phase surface 11a treated, for example, with avidin. This solid-phase surface 11a has been treated with avidin and therefore, is positively charged. As the probe nucleic acid indicated by sign D, such as a DNA probe, is modified at an end thereof with biotin, the probe nucleic acid D is immobilized on the solid-phase surface 11a via an avidin-biotin linkage formed between the detection nucleic acid D and the avidin on the solid-phase surface 11a.

The immobilization method of a probe substance such as the probe nucleic acid D is not limited to relying upon an avidin-biotin linkage, and another method making use of a disulfide linkage or the like can also be used as desired. Accordingly, the solid-phase surface 11a is not particularly limited to the avidin-treated construction.

In this embodiment, the solid-phase surface 11a is arranged on the substrate by way of example. The present invention is not limited to this embodiment and encompasses any critical surface relative to a region or space. The critical surface is capable providing a place of reaction for an interaction, such as surfaces of solid-phase beads.

When the solid-phase surface 11a is positively charged, the nonspecific adsorption of a substance having a negative charge and an intercalator can be prevented by adjusting the concentration of the salt in the salt-containing HEPES buffer. Described specifically, the nonspecific adsorption of substances having a negative charge, such as the probe nucleic acid D and the target nucleic acid T, the intercalator I and the like to the solid-phase surface 11a can be effectively prevented.

The prevention of the nonspecific adsorption of the probe nucleic acid D, the target nucleic acid T, the intercalator I and the like to the solid-phase surface 11a makes it possible to omit or simplify the washing step which is required to remove substances nonspecifically adsorbed on the solid-phase surface 11a. As a consequence, the procedure upon detection of an interaction can be simplified.

When the intercalator I adsorbs nonspecifically to the positively-charged solid-phase surface 11a, the intercalator I emits fluorescence which acts as a background noise. In the present invention, however, the salt-containing HEPES buffer can effectively prevent the nonspecific adsorption of the intercalator I. It is, therefore, possible to make the S/N ratio better and hence, to improve the accuracy of detection of hybridization.

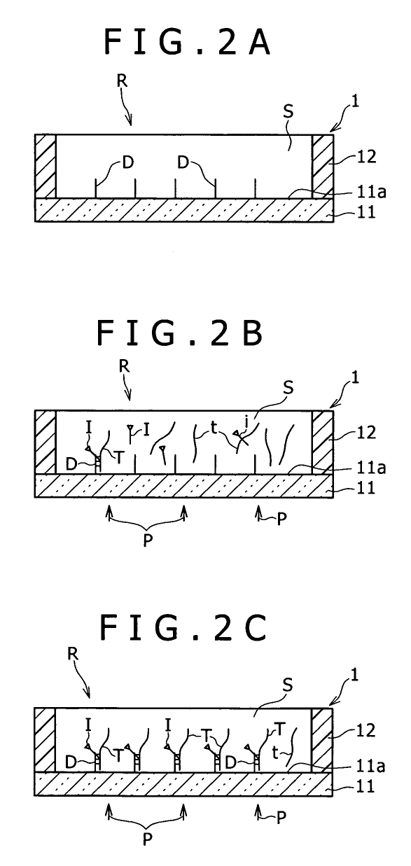

An example of the hybridization detecting method as an embodiment of the interaction detection method according to the present invention will next be described specifically in the order of stages A to C shown in FIGS. 2A to 2C, respectively.

Firstly, FIG. 2A shows the stage A that the salt-containing HEPES buffer S has been added by dropping, injection or the like to the reaction region R with the probe nucleic acid D immobilized therein.

In the present invention, the binding characteristics of the intercalator I can be retained for a long time owing to the use of the salt-containing HEPES buffer S. It is, therefore, possible to concurrently add the intercalator I, for example, upon adding the HEPES buffers to the reaction region R.

At this stage A, the salt-containing HEPES buffer S, the target nucleic acid T and the intercalator I may all be added at the same time to the reaction region R by dropping, injection or the like. In this case, the target nucleic acid T and the intercalator I may be added to the reaction region R by dropping, injection or the like after adding them to the salt-containing HEPES buffers beforehand.

FIG. 2B shows the stage B that the target nucleic acid T has been added to the salt-containing HEPES buffer S already stored or held in the reaction region R. At this stage B, the probe nucleic acid D and the target nucleic acid T are ready to proceed with hybridization.

In the present invention, the binding characteristics of the intercalator I can be retained for a long time by the salt-containing HEPES buffer S. It is, therefore, also possible to concurrently add the intercalator I at this stage B upon dropping, injection or the like of the target nucleic acid T. As a consequence, the procedure which would otherwise be required to separately and independently add the intercalator I to the reaction region R can be omitted, thereby making it possible to simplify the overall procedure.

When the intercalator I already exists in the reaction region R at the stage that the target nucleic acid T is dropped, injected or otherwise added to the salt-containing HEPES buffer S in the reaction region R (in other words, when the intercalator I has already been added to the reaction region R at the stage A), the addition of the target nucleic acid T to the reaction region R makes it possible to proceed with hybridization and intercalation in parallel.

As the hybridization and the intercalation both proceed parallelly in the above case, the hybridization can be detected with time by irradiating the fluorescent excitation light P into the reaction region R from this stage.

Some molecules i of the intercalator I may bind to some molecules t of the target nucleic acid in the form of a single strand to emit fluorescence in some instances (see FIG. 2B). This fluorescence becomes a background noise upon detection of hybridization. However, the use of the salt-containing HEPES buffers can make greater the quantity of fluorescence from the intercalator I upon its binding or the like to a double-stranded nucleic acid (the hybridization product or the like between the probe nucleic acid D and the target nucleic acid T) in comparison with the quantity of fluorescence from the intercalator molecules i upon its binding or the like to the target nucleic acid molecules t in the form of the single strand.

The double-stranded nucleic acid (hybridization) can, therefore, be detected with high accuracy even when the unhybridized, single-stranded, target nucleic acid molecules t still exist in the reaction region R. It is, accordingly, possible to omit or simplify the step which is required to wash off the unhybridized, single-stranded nucleic acid molecules t.

In the present invention, an operation can be conducted to change the temperature condition at the stage B shown in FIG. 2B after the target nucleic acid T has been added by dropping, injection or the like to the salt-containing HEPES buffer S in the reaction region R.

Described specifically, the binding characteristics of the intercalator I can be retained in the present invention owing to the use of the salt-containing HEPES buffer S even when the temperature and/or pH of the buffer S changes. The procedure that controls the interior of the reaction region R to a predetermined temperature can, therefore, be surely performed after the addition of the intercalator I to the reaction region R.

Accordingly, the interior of the reaction region R can be heated to a temperature suited for hybridization, for example, with the intercalator I being allowed to exist in the reaction region R subsequent to its addition by dropping, injection or the like. As a result, the progress of the hybridization can be accelerated so that the detection of the hybridization can be rendered more efficient and more accurate.

FIG. 2C schematically illustrates the stage C that subsequent to the addition of the target nucleic acid T to the reaction region R by dropping, injection or the like, a predetermined time has elapsed and the hybridization has been brought to completion (saturation). At this stage C, the probe nucleic acid D and the target nucleic acid T have already been hybridized with each other. The intercalator I, on the other hand, has been inserted and bound to the site of a base pair of the hybridized, double-stranded nucleic acid (the hybridization product or the like between the probe nucleic acid D and the target nucleic acid T).

At present, it is a common procedure to wash off unnecessary substances from the reaction region R (hereinafter referred to as “to conduct a washing step”) before detecting the hybridization. These unnecessary substances are those which like the unhybridized, single-stranded nucleic acid molecules t, have not hybridized and may act as a factor for a reduction in the detection accuracy. In the present invention, however, the washing step of the unnecessary substances can be omitted because as mentioned above, the use of the salt-containing HEPES buffer S can make greater the quantity of fluorescence from the intercalator T upon its binding or the like to the double-stranded nucleic acid in comparison with the quantity of fluorescence from the intercalator molecules t upon their binding to the target nucleic acid molecules t in the form of the single strand.

As has been described above, the binding characteristics of the intercalator I can be retained for a long time by the salt-containing HEPES buffer S in the present invention, so that the intercalator I can be added to the reaction region R by dropping, injection or the like at any stage before the stage that the hybridization is brought to completion (saturation). Further, the hybridization can be detected at any stage in the present invention insofar as it is after the addition of the target nucleic acid T. Furthermore, the present invention can omit or simplify the washing step which is conducted subsequent to the completion of the hybridization.

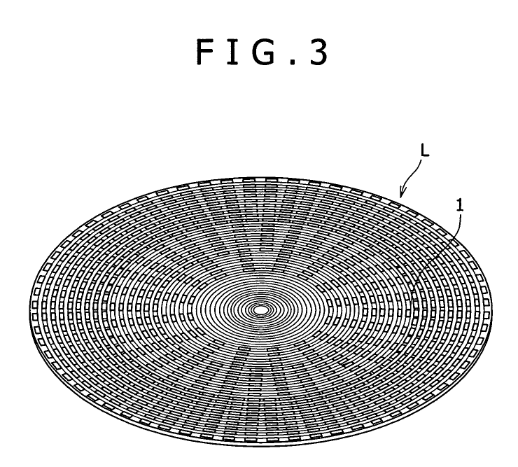

Based on FIG. 3, a description will next be made about the bioassay plate, such as the DNA chip, according to the present invention.

In a bioassay plate L depicted in FIG. 3, a number of interaction detecting units 1 constructed as described above are arranged, for example, in a circular, helical or radial pattern on a substrate in the form of a CD-like disc.

The bioassay plate L can be formed with a similar material as optical information recording media such as CDs (compact discs), DVDs (digital versatile discs) or MDs (mini discs). The plate is not particularly limited in shape to the disc shape shown in FIG. 3, and can be formed into a desired shape depending on its application purpose. The formation of plates with an economical synthetic resin can realize low running cost compared with the glass chips which have been used in related art.

FIG. 4 is a block diagram illustrating one example of the construction of the interaction detecting system (including the hybridization detecting system; hereinafter simply called “the system”) according to the present invention.

The system U illustrated by way of example in FIG. 4 is provided with excitation-light irradiation means 2 capable of irradiating excitation light P into each of the numerous interaction detecting units 1 arranged on the bioassay plate L depicted in FIG. 3, fluorescence detection means 3 capable of reading fluorescence F emitted from the interaction detecting unit 1, heating means 4 capable of heating the medium M (see FIG. 1) stored or held in the reaction region R (see FIG. 1) of the interaction detecting unit 1, temperature control means 5, and a servo control system (not shown) relating to the detection or the like of the, position of the plate.

The system U illustrated by way of example in FIG. 4 is also provided with a disc support 6 for rotatably supporting the bioassay plate L, nozzle heads N capable of dropping or injecting predetermined media M into the interaction detecting unit 1, and a control unit 7 for controlling the system U. For holding and driving the plate L, a similar chucking mechanism as in conventionally-known optical disc drives may also be used.

Into the interaction detecting unit 1, the predetermined media M (M₀ to M₂) can be dropped or injected from the plural nozzle heads N. Examples of the media M include a salt-containing HEPES buffer S (M₀), a probe substance D or target substance T (M₁), and an intercalator I (M₂). The use of the plural nozzle heads N makes it possible to drop or inject predetermined media successively or concurrently at predetermined timing(s) into the reaction region R of the interaction detecting unit 1. The control unit 7 also systematically controls the dropping or injecting operations through the nozzle heads N.

The excitation-light irradiation means 2 is provided with a laser diode 21 for emitting the excitation light P, a collimator lens 22 for converting the excitation light P into parallel rays, dichroic mirrors 23, 24 for reflecting the excitation light P, and a lens 25 for condensing the excitation light P and irradiating it into the interaction detecting unit 1.

Described specifically, the excitation light P is emitted from the laser diode 21, converted into parallel rays through the collimator lens 22, and then refracted at 90° by the dichroic mirror 23. After the parallel rays are further refracted at 90° by the mirror 24, the parallel rays enter the lens 25 supported on an actuator 26 and are then condensed and irradiated through a rear surface of the plate L toward the interaction detecting unit 1. The timing or the like of irradiation of the excitation light is controlled by the control unit 7 (an excitation light control signal 28).

The fluorescence detection means 3 is primarily provided with the detector 31 for sensing the fluorescence F, the lens 32 for condensing the fluorescence F, and a fluorescence-reflecting dichroic mirror 33. The fluorescence detected at the detector 31 is converted by an A/D converter 37 or the like into a predetermined digital signal, which is then fed to the control unit 7 (a fluorescence intensity signal 38).

When the excitation light P is irradiated, fluorescence indicated by sign F is emitted from the intercalator I (see FIG. 1) and the fluorescence F returns to the side of the back surface of the plate L. After the fluorescence F is refracted at 90° by the above-described mirror 24 arranged below the plate L, it penetrates through the excitation-light reflecting dichroic mirror 23, advances straight, and then, is refracted at 90° by the next excitation-light reflecting dichroic mirror 33. Subsequently, the fluorescence F enters the lens 32 and is condensed there, and then, is guided to the detector 31. The fluorescence-reflecting dichroic mirror 33 has a property to reflect the fluorescence F and also transparency to infrared rays to be described subsequently herein.

Florescence is expected to be very weak in intensity compared with RF signals or the like for general optical discs. As the fluorescence-detecting detector 31, it is, therefore, suited to adopt a photomultiplier tube or avalanche photodiode (APD) which has very high sensitivity compared with general photodiodes.

The heating means 4 is provided with an IR laser diode 41 for emitting infrared light Q, a collimator lens 42 for converting the infrared light into parallel rays, a dichroic mirror 43 and the mirror 24 for reflecting the infrared light Q, and the lens 25 for condensing the infrared light Q and irradiating the thus-condensed infrared light into the interaction detecting unit 1.

The infrared light Q is emitted from the laser diode 41, converted into parallel rays through the collimator lens 42, and then refracted at 90° by the dichroic mirror 43. After the parallel rays are further refracted at 90° by the mirror 24, the parallel rays enter the lens 25 and are then condensed and irradiated through the rear surface L1 of the plate L toward the interaction detecting unit 1.

By irradiating the infrared light Q into the interaction detecting unit 1, the medium M stored or held in the interaction detecting unit 1 can be heated, for example, to a temperature suited for the interaction. To control the heating, a method relying upon pulse width modulation (PWM) or pulse amplitude modulation (PAM), for example, may be adopted instead of a method of controlling the irradiation quantity (light quantity) of the infrared light Q or a method of controlling the irradiation time of the infrared light Q.

The temperature control means 5 is provided with a detector 51 and a condenser lens 52. The detector 45, such as a photomultiplier, receives infrared right Q′ reflected back from the interaction detecting unit 1. The condenser lens 52 condenses the infrared light Q′ reflected back from the interaction detecting unit 1.

A portion of the infrared light Q irradiated into the interaction detecting unit 1 by the heating means 4 is reflected back in the interaction detecting unit 1. After the reflected infrared light Q′ is refracted at 90° by the mirror 24 arranged below the plate L, the refracted infrared light penetrates through the excitation-light irradiating dichroic mirror 23, fluorescence-detecting dichroic mirror 33 and infrared-ray irradiating dichroic mirror 43, advances straight, is condensed through the condenser lens 52, and reaches the detector 51.

The reflected infrared light Q′ received at the detector 51 is converted at an A/D converter 57 into a predetermined signal, which is then fed to the control unit 7 (an infrared-ray intensity signal 58). Based on the signal, the heating means 4 is controlled (a temperature control signal 48). As a consequence, the temperature in the reaction region R of the interaction detecting unit 1 is controlled.

In the present invention, the control of the heating conditions can also be performed based on infrared light radiated back from the interaction detecting unit 1. Described specifically, infrared light having been radiated back from the interaction detecting unit 1 and having a wavelength specific to the temperature of the medium M in the interaction detecting unit 1 is received at the detector 51. From a signal (infrared light intensity signal 58) fed from the detector 51, the temperature of the medium M is estimated (temperature control means). Based on the temperature information, the control unit 7 controls the degree of heating by the heating means 4. It is to be noted that the heating means 4 and temperature control means 5 exemplified in FIG. 4 make use of infrared light but these heating means and temperature control means shall not be limited to those relying upon infrared light.

A description will next be made about a preferred embodiment of the “reagent kit” according to the present invention.

The reagent kit according to the present invention is designed to comprise at least one of a salt-containing HEPES buffer, which has been prepared for a detection of an interaction between substances, and a composition useful for the preparation of the salt-containing HEPES buffer. In the case of the composition, it is preferred that the composition permits easy preparation of the salt-containing HEPES buffer by its dilution, dissolution or the like with distilled water.

When reagents useful upon production of bioassay plates such as DNA chips are combined into a kit, the kit may be designed to include, for example, avidin, biotin and an enzyme for modifying a probe nucleic acid with biotin, or compositions formed by concentration, drying or the like of the above-described reagents, in addition to the salt-containing HEPES buffer.

When reagents useful upon detection of hybridization are combined into a kit, the kit may be designed to include, for example, a reagent for preparing a target nucleic acid (an enzyme required upon extraction of a DNA, mRNA or the like from a tissue, cells or the like, an enzyme required for the synthesis of a cDNA, or the like) and an intercalator, or compositions formed by concentration, drying or the like of the above-described reagents, in addition to the salt-containing HEPES buffer.

### EXAMPLE 1

Buffers suited for the structural stability of an intercalator were verified by thin-layer chromatography. As the intercalator, “SYBRGreen I Nucleic Acid Gel Stain” (trade name, product of Cambrex Corporation; “SYBR” is a registered trademark of Molecular Probes, Inc.; hereinafter referred to as “SYBRGreen I”) was used. “SYBRGreen I” is a high-sensitivity color-developing reagent useful for the detection of double-stranded nucleic acids. “SYBRGreen I” exhibits sensitivity as high as 25 to 100 times of ethidium bromide, it is a composition considered to be adequate as an intercalator. It is to be noted that “SYBRGreen I” is labeled with a fluorescent colorant and permits the detection of its binding to a double-bonded nucleic acid on the basis of fluorescence. The experimental procedure will be described next.

Firstly, SYBRGreen I was diluted tenfold with the buffers shown in Table 1 to provide experimental samples (Sample 1 to Sample 7). For the preparation of the HEPES buffers, “HEPES Buffer Solution” (trade name; distributed by Wako Pure Chemical Industries, Ltd. and manufactured by DOJINDO LABORATORIES) was used (the same HEPES buffer solution was used hereinafter).

TABLE 1

| Sample | Sample used for the dilution of SYBRGreen I |
|---|---|
| Sample 1 | 0.1 M HEPES, MgCl₂ 20 mM, pH 7.4 |
| Sample 2 | 0.1 M HEPES, MgCl₂ 20 mM, pH 7.1 |
| Sample 3 | 0.1 M HEPES, MgCl₂ 20 mM, pH 6.8 |
| Sample 4 | PBS, NaCl 250 mM, pH 7.1 |
| Sample 5 | Phosphate buffer, MgCl₂ 10 mM, pH 7.1 |
| Sample 6 | Tris-EDTA buffer (preparation) |
| Sample 7 | DMSO (preparation) |

After the samples were left over at room temperature for a predetermined time, thin-layer chromatography was performed. The thin-layer chromatography was performed in accordance with the following specific procedure. Firstly, the solutions prepared with Samples 1 to Sample 7, respectively, were spotted side by side at suitable intervals therebetween at positions of the same height from the lower end of a thin-layer plate (in this experiment, a plastic plate with silica coated as a solid-phase carrier thereon was used), and were then dried in air. A lower end portion of the thin-layer plate on which the individual samples had been spotted was immersed in a developing solution (DMSO) to develop them at room temperature for 10 minutes, and the resulting migrations of the spots were observed.

As a result, with the samples making use of the buffers containing 0.1 M HEPES and 20 mM MgCl₂ (Samples 1 to 3), even when spotted after having been left over for 5 hours, about 80% of their spots all remained at the same height as the spots of the preparations (Sample 6, Sample 7), and no substantial migrations of the spots were observed. Accordingly, SYBRGreen I is not considered to have undergone any substantial structural change in those buffers.

In contrast, with Sample 4, even when spotted after having been left over at room temperature for 15 minutes subsequent to the dilution of SYBRGreen I with the buffer, practically all the spots had migrated to heights different from the spots of the preparations (Sample 6, Sample 7). With Sample 5, even when spotted after having been left over at room temperature for 2 hours subsequent to the dilution, practically all the spots had migrated to heights different from the spots of the preparations (Sample 6, Sample 7). Accordingly, SYBRGreen I is considered to have undergone substantial structural changes in those buffers, respectively.

The above results indicate that a buffer containing 0.1 M HEPES and 20 mM MgCl₂ is most suited for the retention of the structural stability of SYBRGreen I. In other words, it is considered that the use of a buffer containing 0.1 M HEPES and 20 mM MgCl₂ makes it possible to stably retain the structure of SYBRGreen I for a long time and can prohibit changes in the binding characteristics of SYBRGreen I to a double-stranded nucleic acid.

A comparison was also made among Samples 1 to 3. Even when the buffer used was the one having pH 6.8, no substantial spot migrations were observed as in the case of the buffers of pH 7.1 and pH 7.4. It has, therefore, been ascertained that the use of a salt-containing HEPES buffer makes it possible to sufficiently retain the structural stability of SYBRGreen I even under the pH condition of 6.8 which is lower than the recommended range for SYBRGreen I (pH 7.5 to pH 8.0).

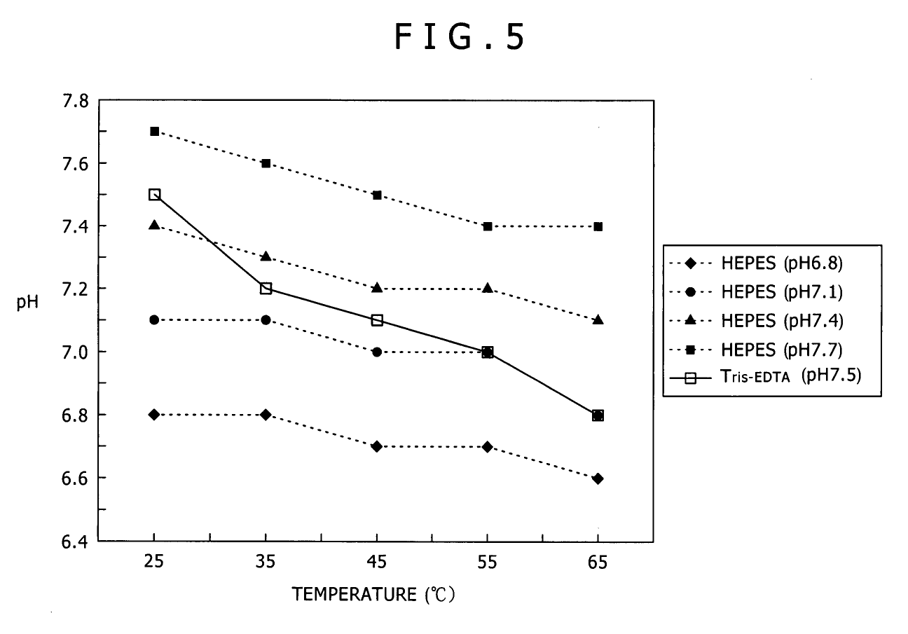

In general, the pH of a buffer drops as the temperature rises. The present inventors investigated changes in the pHs of HEPES buffers at varied temperatures. In the case of the HEPES buffer of pH 7.7 at room temperature (25° C.), the pH dropped to 7.4 at 55° C., and in the case of the HEPES buffer of pH 7.1 at room temperature (25° C.), the pH dropped to 7.0 at 55° C. (see FIG. 5). It is, therefore, suggested that the use of a salt-containing HEPES buffer, the pH of which has been adjusted to pH 7.1 or higher at room temperature, makes it possible to stably retain the structure of an intercalator in the buffer even when the temperature of the buffer is adjusted, for example, to a temperature suited for annealing.

### EXAMPLE 2

An investigation was conducted about the relationship between the temperature and pH of a buffer.

Provided were HEPES buffers, the pHs of which were 6.8, 7.1, 7.4 and 7.7, respectively, at room temperature (25° C.), and a Tris-EDTA buffer (pH 7.5). The pHs of the individual buffers were measured at temperatures of 35° C., 45° C., 55° C. and 65° C. The results are shown in FIG. 5.

As shown in FIG. 5, the Tris-EDTA buffer significantly varied in pH as the temperature changed. The HEPES buffers, on the other hand, underwent only small variations in pH even when their temperatures arose.

An intercalator may be impaired in its binding characteristics to a double-stranded nucleic acid and the quantity of fluorescence upon its binding to the double-stranded nucleic acid even by a very small change in the conditions. The above-described experimental results indicate that the use of a HEPES buffer makes it possible to control a pH change small even when conditions involving a change in temperature are applied. It is, therefore, suggested that, even when conditions involving a change in temperature are applied, an intercalator having sufficient binding characteristics and fluorescence quantity, for example, under the conditions that a buffer shows an acidic value of pH 6.8 at 25° C. can be prevented from a pH-dependent, functional deterioration or modification and can retain its binding characteristics to a double-stranded nucleic acid and the quantity of fluorescence upon its binding to the double-stranded nucleic acid.

### EXAMPLE 3

Verification was conducted on buffers capable of making greater the binding ratio of the binding between an intercalator and a double-stranded nucleic acid to the binding between the intercalator and a single-stranded nucleic acid. The following experimental procedure was followed.

Firstly, the buffers shown in Table 2 were provided.

TABLE 2

| Sample | Buffer used for the dilution of SYBRGreen I |
|---|---|
| Sample 1 | 0.1 M HEPES, MgCl₂ 20 mM, pH 7.7 |
| Sample 2 | Phosphate buffer, NaCl 200 mM, pH 7.15 |
| Sample 3 | Tris-EDTA buffer, NaCl 200 mM, pH 7.8 |
| Sample 4 | Tris-EDTA buffer, MgCl₂ 20 mM, pH 7.5 |

Next, SYBRGreen I was diluted 10,000-fold with the buffers, respectively. A single-stranded or double-stranded nucleic acid of 30 mer was added at 100 nM and, after having been left over at room temperature for 10 minutes or 30 minutes, fluorometry was performed. The fluorometric results of those left over at room temperature for 10 minutes are shown in Table 3, and the fluorometric results of those left over at room temperature for 30 minutes are shown in Table 4.

TABLE 3

| | Fluorescence | Fluorescence | |
|---|---|---|---|
| | value (A) | value (B) | |
| | when the | when the | |
| | single- | double- | |
| Sample (left | stranded | stranded | Fluorescence |
| over for 10 | nucleic acid | nucleic acid | ratio, |
| minutes) | was added | was added | (A)/(B) |
| Sample 1 | 703.8 | 9330.2 | 1:13.3 |
| Sample 2 | 815.7 | 7928.1 | 1:9.7 |
| Sample 3 | 881.2 | 8997.1 | 1:10.2 |
| Sample 4 | 768.4 | 7718.0 | 1:10.0 |

TABLE 4

| | Fluorescence | Fluorescence | |
|---|---|---|---|
| | value (A) | value (B) | |
| | when the | when the | |
| | single- | double- | |
| Sample (left | stranded | stranded | Fluorescence |
| over for 30 | nucleic acid | nucleic acid | ratio, |
| minutes) | was added | was added | (A)/(B) |
| Sample 1 | 747.8 | 9116.7 | 1:12.2 |
| Sample 2 | 687.7 | 6571.8 | 1:9.56 |
| Sample 4 | 850.8 | 6387.8 | 1:7.51 |

As shown in Table 3 and Table 4, the use of the buffer containing 0.1 M HEPES and 20 mM MgCl₂ (Sample 1) was able to make greater the fluorescence ratio of the fluorescence upon addition of the double-stranded nucleic acid to the fluorescence upon addition of the single-stranded nucleic acid in comparison with the other buffers (Samples 2 to 4). Further, the buffer containing 0.1 M HEPES and 20 mM MgCl₂ (Sample 1) was also able to make greater the fluorescence value itself upon addition of the double-stranded nucleic acid in comparison with the other buffers (Samples 2 to 4).

This firstly indicates that the use of a buffer containing 0.1 M HEPES and 20 mM MgCl₂ makes it possible to improve the accuracy of measurement upon detection of hybridization. Described specifically, the hybridized, double-stranded nucleic acid can be detected at higher accuracy because it was possible to make greater the fluorescence ratio of the fluorescence upon addition of the double-stranded nucleic acid to the fluorescence upon addition of the single-stranded nucleic acid (in other words, the binding ratio of the binding between the intercalator and the double-stranded nucleic acid to the binding between the intercalator and the single-stranded nucleic acid).

It is also indicated that the use of a buffer containing 0.1 M HEPES and 20 mM MgCl₂ makes it possible to omit or simplify the procedure required to wash of the unhybridized, single-stranded nucleic acid molecules. This feature will next be described specifically.

When hybridization is detected by a DNA chip or the like, a single-stranded nucleic acid is not used in its entirety for the hybridization, and the unhybridized, single-stranded nucleic acid molecules remain in the buffer. An intercalator reacts not only with a double-stranded nucleic acid but also some molecules of the single-stranded nucleic acid, so that the existence of the unhybridized, single-stranded nucleic acid molecules becomes a noise upon detection of the hybridized, double-stranded nucleic acid. Therefore, a step has been needed to wash off the unhybridized, single-stranded nucleic acid.

In contrast, it was still possible to detect the hybridized, double-stranded nucleic acid in this experiment despite the co-existence of the unhybridized, single-stranded nucleic acid in the reaction region because the use of the buffer containing 0.1 M HEPES and 20 MM MgCl₂ was able to make greater the binding ratio of the binding between the intercalator and the double-stranded nucleic acid to the binding between the intercalator and the single-stranded nucleic acid. Hybridization can, therefore, be detected, for example, even when a single-stranded nucleic acid as a target substance and an intercalator are dropped, injected or otherwise added at the same time to the buffer. It is, accordingly, possible to omit or simplify the procedure that washes off the unhybridized, single-stranded nucleic acid.

Table 3 and Table 4 were next compared with each other. When the buffer containing 0.1 M HEPES and 20 mM MgCl₂ (Sample 1) was used, the fluorescence value upon addition of the double-bonded nucleic acid did not decrease practically even when the buffer was left over at room temperature for 30 minutes. With the other buffers (Samples 2 and 4), however, the fluorescence values decreased by 1,000 or more.

This indicates that the use of a buffer containing 0.1 M HEPES and 20 mM MgCl₂ can maintain the binding between an intercalator and a double-bonded nucleic acid for a long time without any substantial unbinding.

It is, therefore, possible to reduce a decrease in the bound amount due to a difference in the time elapsed from the dropping, injection or the like of the intercalator to the buffer until the detection of hybridization, or to reduce differences in the bound amount among samples. In addition, hybridization can still be detected stably even when a predetermined time has elapsed from the addition of the intercalator to the reaction region until the detection of the hybridization.

### EXAMPLE 4

Verification was conducted on buffers capable of retaining the stability of an intercalator even when high-temperature conditions are applied. The following experimental procedure was followed.

Firstly, the buffers shown in Table 5 were provided.

TABLE 5

| Sample | Buffer used for the dilution of SYBRGreen I |
|---|---|
| Sample 1 | 0.1 M HEPES, MgCl₂ 20 mM, pH 7.7 |
| Sample 2 | Tris-EDTA buffer, MgCl₂ 20 mM, pH 7.5 |

Next, SYBRGreen I was diluted 10,000-fold with the buffers, respectively. A single-stranded or double-stranded nucleic acid of 30 mer was added at 100 nM. Conditions of 95° C. for 5 minutes, 10 minutes or 15 minutes were applied, the thus-obtained media were chilled back to room temperature, and then, fluorometry was performed. The fluorometric results of those left over at 95° C. for 5 minutes are shown in Table 6, the fluorometric results of those left over at 95° C. for 10 minutes are shown in Table 7, and the fluorometric results of that left over at 95° C. for 15 minutes are shown in Table 8.

TABLE 6

| | Fluorescence | Fluorescence | |
|---|---|---|---|
| | value (A) | value (B) | |
| | when the | when the | |
| | single- | double- | |
| Sample (left | stranded | stranded | Fluorescence |
| over for 5 | nucleic acid | nucleic acid | ratio, |
| minutes) | was added | was added | (A)/(B) |
| Sample 1 | 803.26 | 7315.7 | 1:9.1 |
| Sample 2 | 714.14 | 6427.8 | 1:9.0 |

TABLE 7

| | Fluorescence | Fluorescence | |
|---|---|---|---|
| | value (A) | value (B) | |
| | when the | when the | |
| | single- | double- | |
| Sample (left | stranded | stranded | Fluorescence |
| over for 10 | nucleic acid | nucleic acid | ratio, |
| minutes) | was added | was added | (A)/(B) |
| Sample 1 | 772.6 | 7438.4 | 1:9.6 |
| Sample 2 | 622.6 | 5440.2 | 1:8.7 |

TABLE 8

| | Fluorescence | Fluorescence | |
|---|---|---|---|
| | value (A) | value (B) | |
| | when the | when the | |
| | single- | double- | |
| Sample (left | stranded | stranded | Fluorescence |
| over for 15 | nucleic acid | nucleic acid | ratio, |
| minutes) | was added | was added | (A)/(B) |
| Sample 1 | 785.2 | 7837.8 | 1:10.0 |

As shown in Table 6, Table 7 and Table 8, the use of the buffer containing 0.1 M HEPES and 20 mM MgCl₂ (Sample 1), despite the application of the high-temperature conditions, gave about 1:10 as the fluorescence ratio of the fluorescence upon addition of the single-stranded nucleic acid to the fluorescence upon addition of the double-stranded nucleic acid, and was able to obtain the sufficiently large fluorescence ratio. Further, the fluorescence value itself upon addition of the double-stranded nucleic acid was greater than 7,000 despite the application of the high-temperature conditions, and remained higher in comparison with the other buffers (Samples 2 to 4).

This indicates that the use of the buffer containing 0.1 M HEPES and 20 mM MgCl₂ (Sample 1) can retain the stability of an intercalator even when high-temperature conditions are applied. It is, therefore, possible to retain the binding characteristics of the intercalator to a double-stranded nucleic acid and the quantity of fluorescence upon its binding to the double-stranded nucleic acid, for example, even when the temperature of the buffer is adjusted subsequent to the dropping, injection or the like of the intercalator to the buffer.

### EXAMPLE 5

This example demonstrates that the inclusion of a salt in a HEPES buffer can prevent the nonspecific adsorption of nucleic acid molecules to a positively-charged solid-phase surface in a reaction region. The following experimental procedure was followed.

Firstly, a HEPES buffer containing 0.1 M HEPES and 20 mM MgCl₂ and having pH 7.1 was prepared. In addition, a quartz crystal resonator with its Au electrode surfaces coated with avidin was provided. As the avidin, the product of Wako Pure Chemical Industries, Ltd. (trade name: “Avidin”) was used (the same avidin was used hereinafter). The coating of avidin on the Au electrode surfaces was conducted following the process described in the protocol of a QCM instrument for the quantitation of biomolecular interactions employed in this experiment [“AFFINIX Q”] (Model: “QCM2000”, trade name; quartz crystal resonation system, resonant frequency: 27 MHz; manufactured by Initium Inc.; hereinafter referred to as “the QCM instrument”; the same QCM was used hereinafter).

In a reaction bath of the QCM instrument, the salt-containing buffer shown in Table 9 was placed, together with and a 30-mer oligonucleotide (30 nM), and the quartz crystal resonator was soaked in the reaction bath. A change (ΔF, unit: Hz) in the frequency of the quartz crystal resonator was measured to determine the amount of the 30-mer oligonucleotide adsorbed on the avidin layers of the quartz crystal resonator. It is to be noted that as the 30-mer oligonucleotide, one custom-synthesized by ESPEC OLIGO SERVICE CORP. was used. The results are shown in Table 9.

TABLE 9

| | | Adsorption on the avidin |
|---|---|---|
| Buffer | ΔF (Hz) | layers (ng) |
| HEPES buffer + MgCl₂ | 11 | 0.3 |

To immobilize a probe nucleic acid in each reaction region on a plate, for example, via an avidin-biotin linkage, a positively-charged solid-phase surface (avidin layer) is arranged in the reaction region. On the other hand, a target nucleic acid to be hybridized with the probe nucleic acid is a substance having a negative charge and therefore, nonspecifically adsorbs on the positively-charged solid-phase surface.

As a result of this experiment, it has been found that as shown in Table 9, the use of a salt-containing HEPES buffer can prevent the nonspecific adsorption of a target nucleic acid on a positively-charged solid-phase surface.

As the prevention of the nonspecific adsorption of the target nucleic acid to the solid-phase surface makes it possible to omit the procedure which would otherwise be required to wash off the nonspecifically-adsorbed target nucleic acid, the procedure can be simplified upon detection of hybridization. Further, the successful prevention of the nonspecific adsorption of the target nucleic acid to the solid-phase surface can also inhibit the production of a noise, thereby making it possible to improve the accuracy of a detection of hybridization.

### EXAMPLE 6

This example demonstrates that the nonspecific adsorption of an intercalator can be prevented by adjusting the concentration of a salt in a HEPES buffer. The following experimental procedure was followed.

Firstly, a HEPES buffer containing 0.1 M HEPES and 20 mM MgCl₂ and having pH 7.1 was prepared. Similar to Example 4, a quartz crystal resonator with its Au electrode surfaces coated with avidin was also provided.

In the reaction bath of the QCM instrument, the buffer and SYBRGreen I (intercalator) were placed, and the quartz crystal resonator was soaked in the reaction bath. A change (ΔF, unit: Hz) in the frequency of the quartz crystal resonator was measured to determine the amount of SYBRGreen I adsorbed on the avidin layers of the quartz crystal resonator. It is to be noted that as SYBRGreen I, one prepared by diluting 10,000-fold the dilution recommended in the protocol was used. The results are shown in Table 10.

TABLE 10

| | | Adsorption on the avidin |
|---|---|---|
| Buffer | ΔF (Hz) | layers (ng) |
| HEPES buffer + MgCl₂ | 0 | 0 |

As a result, no substantial adsorption of SYBRGreen I on the avidin layers was observed in the salt-containing HEPES buffer as shown in Table 10. It has, therefore, become evident from this experiment that, even in the case of a positively-charged solid-phase surface such as the avidin layers, an adequate adjustment of the concentration of a salt in a buffer can prevent the nonspecific adsorption of not only a target nucleic acid but also an intercalator to the positively-charged solid-phase surface.

This also indicates the feasibility of quantitation of hybridization by the use of an intercalator. As the nonspecific adsorption of an intercalator to a solid-phase surface can be prevented by adequately adjusting the concentration of a salt in a buffer, signals (noise) based on the nonspecific adsorption are not produced. It is, therefore, possible to detect only hybridization-based signals. It is, accordingly, considered possible to make an improvement in measurement accuracy and also to achieve a quantitative detection of hybridization on the basis of the intensity of signals. Even when an intercalator has reacted with some unhybridized molecules of a single-stranded nucleic acid, a quantitative detection of the hybridization is still feasible because the intensity of signals can be adjusted by subtracting the background noise.

It should be understood by those skilled in the art that various modification, combinations, sub-combinations and alterations may occur depending on design requirements and other factors insofar as they are within the scope of the appended claims or the equivalents thereof.

What is claimed is:

1. A method for detecting an interaction between substances, which comprises allowing a HEPES buffer, which contains a salt, to exist in a reaction region that provides a place of interaction for said interaction.

2. The method according to claim 1, wherein a concentration of said salt in said salt-containing HEPES buffer is adjusted such that a substance having a negative charge can be prevented from undergoing non-specific adsorption on a positively-charged surface of a solid phase.

3. The method according to claim 2, wherein said substance having said negative charge comprises at least one of a probe nucleic acid and a target nucleic acid.

4. A method for detecting hybridization between a probe nucleic acid and a target nucleic acid by using an intercalator, which comprises allowing a HEPES buffer, which contains a salt, to exist in a reaction region that provides a place of hybridization for said hybridization.

5. The method according to claim 4, wherein said salt-containing HEPES buffer has such a salt concentration as giving an ionic strength sufficient to prevent nonspecific adsorption of a substance, which has a negative charge, to a positively-charged solid-phase surface.

6. The method according to claim 5, wherein said salt-containing HEPES buffer has such a salt concentration as giving an ionic strength equivalent to at least 20 mM of MgCl₂.

7. The method according to claim 4, further comprising a procedure to control a temperature in said reaction region, in which said salt-containing HEPES buffer and said intercalator are allowed to exist, at a level suited for said hybridization.

8. The method according to claim 4, wherein said salt-containing HEPES buffer has an acidic pH.

9. The method according to claim 4, wherein said intercalator is added to said reaction region at one of the following stages (1) to (3):
(1) a pre-stage at which said target nucleic acid is added to said salt-containing HEPES buffer in said reaction region,
(2) a stage at which said target nucleic acid is added to said salt-containing HEPES buffer in said reaction region, and
(3) a stage at which said hybridization proceeds.

10. An interaction detecting unit comprising a reaction region for providing a place of interaction for an interaction between substances, and a medium existing in said reaction region and comprising a salt-containing HEPES buffer.

11. A bioassay plate comprising a number of interaction detecting units according to claim 10.

12. An interaction detecting system comprising at least, temperature control means for controlling a temperature in said reaction region of an interaction detecting unit according to claim 10, and means for detecting said interaction between said substances.

13. A hybridization detecting unit comprising a reaction region for providing a place of hybridization for hybridization between a probe nucleic acid and a target nucleic acid, and a medium existing in said reaction region and comprising a salt-containing HEPES buffer and an intercalator.

14. The hybridization detecting unit according to claim 13, wherein said reaction region has a construction such that said reaction region can be controlled at a temperature suited for said hybridization.

15. The hybridization detecting unit according to claim 13, wherein said salt-containing HEPES buffer has an acidic pH.

16. A DNA chip comprising a number of hybridization detecting units according to claim 13.

17. A hybridization detecting system comprising at least, temperature control means for controlling a temperature in said reaction region of a hybridization detecting unit according to claim 13, and fluorescence detecting means for having said intercalator fluorescently excited to emit fluorescence and detecting an intensity of said fluorescence.

18. A reagent kit comprising at least one of a salt-containing HEPES buffer, which has been prepared for a detection of an interaction between substances, and a composition useful for the preparation of said salt-containing HEPES buffer.

19. The reagent kit according to claim 18, wherein said interaction is hybridization.

20. The reagent kit according to claim 19, further comprising an intercalator for a detection of said hybridization.

21. An interaction detecting system comprising at least, a temperature control section for controlling a temperature in said reaction region of an interaction detecting unit according to claim 10, and a section for detecting said interaction between said substances.

22. A hybridization detecting system comprising at least, a temperature control section for controlling a temperature in said reaction region of a hybridization detecting unit according to claim 13, and a fluorescence detecting section for having said intercalator fluorescently excited to emit fluorescence and detecting an intensity of said fluorescence.
